(19) United States
(12) Patent Application Publication
(10) Pub. No.: US 2009/0216258 A1
(43) Pub. Date: Aug. 27, 2009

(54) MEDICAL HAND ATTACHMENT

(75) Inventor: Volker Geuder, Heidelberg (DE)

Correspondence Address: MOORE & VAN ALLEN PLLC, P.O. BOX 13706, Research Triangle Park, NC, 27709, US

(73) Assignee: Geuder AG, Heidelberg (DE)

(21) Appl. No.: 12/090,582

(22) PCT Filed: Oct. 15, 2007
(86) PCT No.: PCT/DE2007/001809

§ 371 Date: Dec. 11, 2008

(30) Foreign Application Priority Data
Oct. 18, 2006 (DE) 10 2006 049 702.3

(51) Int. Cl. A61B 17/32

(52) U.S. Cl. 606/170

(57) ABSTRACT

A hand-held medical instrument, in particular a surgical cutting instrument, preferably for use in ophthalmology, with a housing that serves a grip, and with a tool holder that is provided in particular for a knife and is arranged so as to be displaceable in the housing via an actuation member, tool being able to be displaced from a storage position in the housing to a working position outside of the housing by actuation of the actuating member, is characterized in that the housing has openings in the housing wall, at least in the area of the tool located in the storage position.

The invention relates to a hand-held medical instrument, in particular a surgical cutting instrument, preferably for use in ophthalmology, with a housing serving as a grip, and with a tool holder that is provided in particular for a knife and is arranged so as to be displaceable in the housing via an actuating member, the tool being able to be displaced from a storage position in the housing to a working position outside of the housing by actuation of the actuating member.

Hand-held medical instruments are known in a wide variety of designs from medical practice. An important aspect is that the tool, generally held by a tool holder, can be moved from an outer working position to a storage position inside the housing.

The hand-held instruments of the type in question that are known from practice are problematic in terms of cleaning, since the soiled tool is located in the storage position in the housing. Inside the housing, however, the tool is only accessible to a limited extent. If, for cleaning purposes, the tool were to be left in the working position outside of the housing, there would be a danger of damaging it or of injuring the person handling it. This is especially so if the tool is a diamond blade adhesively bonded into the tool holder. There is then not only a danger of damage, but also a danger of loss of the expensive blade.

Hand-held instruments of the type in question here were previously cleaned in ultrasound baths and then sterilized.

It has in the meantime been found that the cleaning can be done much more easily in suitable washing machines. However, the hand-held instruments of the type in question are not really suitable for such cleaning, especially in light of the problems outlined above. Particularly if an unprotected tool were to be cleaned in a washing machine, there would be an increased danger of damage or loss of the blade, with the result that the cleaning method, although simple in itself, cannot really be implemented satisfactorily in the washing machine.

As prior art, reference is made solely by way of example to EP 0 870 472 A1.

The object of the present invention is therefore to design and develop a hand-held medical instrument of the type in question, in such a way that it is suitable for automatic cleaning in a washing machine, without danger to the tool.

According to the invention, the above object is achieved by a hand-held medical instrument having the features of patent claim 1. In accordance with the latter, the hand-held instrument in question is characterized in that the housing has openings in the housing wall, at least in the area of the tool located in the storage position.

According to the invention, it has been recognized that hand-held medical instruments of the type in question here are suitable for cleaning in washing machines only if special design measures are taken. Thus, the housing has openings in the housing wall, at least in the area of the tool located in the storage position, such that the spraying or cleaning jet in the washing machine reaches the soiled tool. In other words, the cleaning of the tool takes place in the storage position of the tool, the openings in the housing wall allowing the cleaning jet to exert its action. Since the openings in question are spatially limited openings, the spraying jet, for example emanating from a rotating spray arm, is broken up regularly, such that to this extent there is a kind of neutralizing of the spraying jet, but without its effect being annulled.

According to the invention, it has therefore been found that the tool can be readily cleaned in its storage position, i.e. inside the housing of the hand-held instrument, if the housing wall is perforated or provided with openings. When the tool has been driven in, i.e. with the tool located in the storage position, the tool is sufficiently well protected and the danger of injury is excluded. Thus, the housing satisfies its protective function, as before, but permits cleaning of the tool from the outside. Accordingly, the hand-held instrument according to the invention is very particularly suitable for automatic cleaning, in particular in a rinsing or washing machine specially provided for this purpose.

It has already been stated above that the openings are to be provided at least in the area of the tool located in the storage position, to ensure that sufficiently good cleaning of the tool is possible through the openings. To ensure complete cleaning of the hand-held instrument, specifically from outside and from inside, it is advantageous if the housing has openings in the housing wall along its entire length. In this way, it is not just the tool, but the entire mechanism within the hand-held instrument that can be cleaned and then sterilized.

In concrete terms, the housing has a housing tip tapering preferably continuously as far as the free end, and a preferably cylindrical housing shaft. To this extent, the housing is similar to the housing of a ballpoint pen. The openings can be arranged both in the housing tip and also in the housing shaft. The storage position of the tool is in such a case located in the housing tip, such that the openings located there are of very particular importance.

The openings are also advantageously formed all the way round the housing, i.e. about the entire circumference of the housing. It is also conceivable for the openings to be offset relative to one another in the axial direction or to be aligned one behind another.

As regards the geometrical design of the openings, it will be noted at this point that they can have any desired shapes. Particularly with a view to safe handling, and if at all possible to avoid injuries in the area of the openings, the latter can be made round or have rounded edge areas. A slit-like design, preferably of oval shape, is advantageous in allowing relatively large openings to be created, and, with a suitable number of such openings, it is possible for the housing to be formed as a kind of housing cage. The cage-like construction protects the interior, in particular the tool, and also protects the user against injury. The tool is sufficiently well secured in the storage position by the housing cage. Automatic cleaning is possible, especially as the spraying or cleaning jet, although broken up, passes into the housing and as far as the tool and can exert a sufficiently good cleaning action.

It is also advantageous if the housing, as far as the housing tip, has a cylindrical passage in which a plunger carrying the tool holder is displaceably arranged. The plunger could have a continuously uniform design. Advantageously, the plunger tapers toward the tool holder, preferably via a kind of step formation. In the area of the taper, i.e. between the plunger and the housing, a compression spring is advantageously arranged against whose force the tool can be pushed into the working position. The compression spring thus acts between the plunger and the housing, such that, in order to extract the tool, it is necessary to overcome the pretension defined by the compression spring. Here too, there is a similarity to the actuation of a conventional ballpoint pen.

The plunger used to actuate the tool is guided in the cylindrical area of the housing. The tapered area of the plunger, which extends as far as the housing tip, is advantageously guided in the transition between the cylindrical area of the housing and the housing tip or in the housing tip itself. When actuated, the tapered area of the plunger can be pushed out of the housing tip, such that the tool is located in the exposed working position.

It is very particularly advantageous if the plunger is assigned a preferably orthogonally protruding guide pin, which runs in a guide formed in the housing. This guide is to be understood as a notch or recess in the housing wall. The guide pin can be welded onto the plunger or screwed into the plunger.

The guide provided for the guide pin is specifically designed as a slit in the housing wall. To avoid inadvertent actuation, in particular to avoid the tool inadvertently being pushed out into the working position, the slit comprises a rear catch, in the form of a niche forming an abutment. A front catch can also be provided against the tool inadvertently being pushed or driven back into the storage position, which front catch is designed in a manner corresponding to the catch against inadvertent pushing out of the tool, but with a reverse orientation.

By turning the plunger, preferably via the actuating member, the guide pin can be turned, in a suitable axial position, out of the locked position and into the locked position. This means that an axial actuation is not sufficient to push the tool out and drive it back in. For safety, an additional turning of the plunger via the actuating member is needed, for which purpose it is expedient to provide the actuating member with a roughened surface or the like that ensures a firm grip.

As regards the design of the actuating member, it is also advantageous if the latter is made thicker or widened relative to the plunger, preferably to the size of the housing. Despite a roughened surface or the like to facilitate handling, it is conceivable for the actuating member to have flattened areas at opposite ends. To turn the plunger, the actuating member can be gripped at these areas such as to permit a locking or unlocking.

It will further be noted that the plunger can be moved counter to the force of the spring already mentioned above. On the basis of this action of force, it is conceivable for the slits serving as guides to be provided with beveled end surfaces, such that the guide pin moves as it were automatically into the respective locking position, such that an unlocking or turning of the plunger via the actuating member is always necessary to allow repeated actuation. This structural measure affords a considerable degree of operating safety.

The plunger, the tapered area of the plunger, the actuating member and the tool holder can be made in one piece. Multi-part designs, for example also using different materials, are also conceivable.

It is also important that the tool holder comprises a seat for the tool, into which seat the tool is inserted, as always. In the simplest case, the tool is plugged or adhesively bonded into the seat, particularly if the tool is a miniaturized diamond blade.

The housing, the plunger, the actuating member and the tool holder can be made of different materials, it being advantageous for the structural parts to be made of stainless steel and/or of titanium or a titanium alloy. Sufficient flexural strength is afforded by such materials, and these materials are also particularly suitable for automatic cleaning in washing machines using suitable cleaning agents, specifically because of the material-specific resistance.

There are now various possible ways in which the teaching of the present invention can be advantageously embodied and developed. Reference is made, on the one hand, to the patent claims dependent on patent claim 1 and, on the other hand, to the following explanation of an illustrative embodiment of the invention based on the drawing. In conjunction with the explanation of the preferred illustrative embodiment of the invention based on the drawing, generally preferred embodiments and developments of the teaching are also explained. In the drawing:

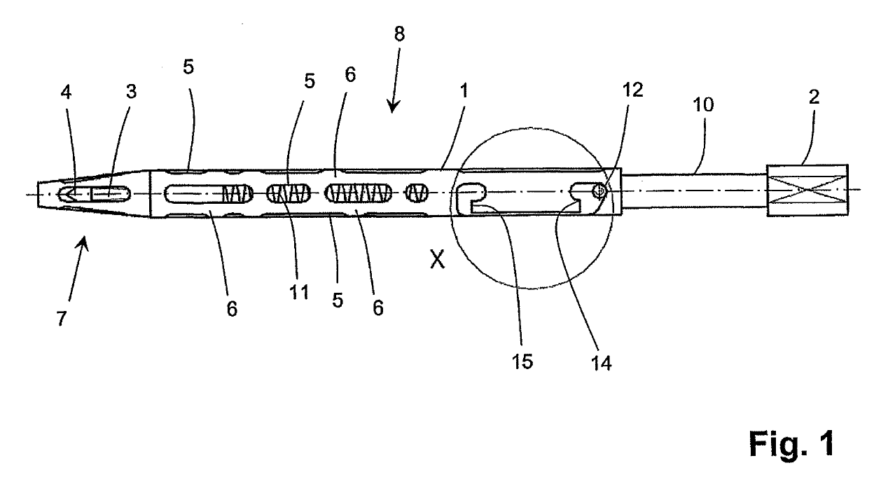

FIG. 1 shows a schematic view of an illustrative embodiment of a hand-held medical instrument according to the invention, the instrument being a cutting instrument equipped with a diamond blade and used in ophthalmology, and the tool being located in the retracted storage position,

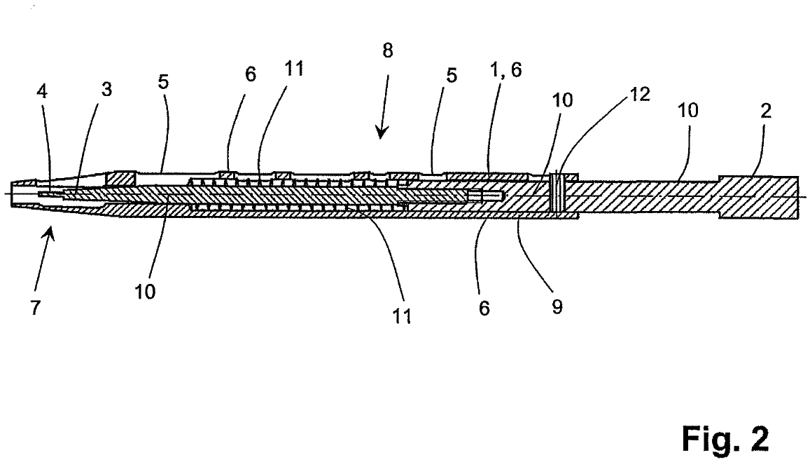

FIG. 2 shows a schematic view of the article from FIG. 1, in cross section and rotated through 90° about the longitudinal axis relative to FIG. 1,

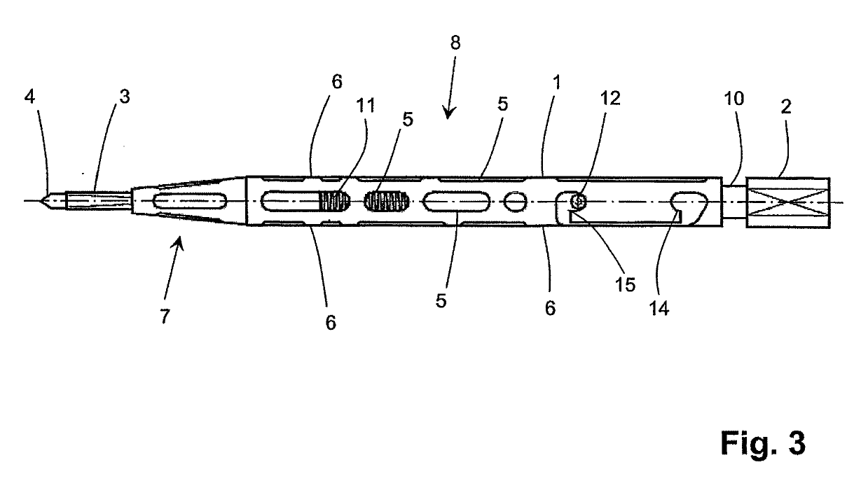

FIG. 3 shows a schematic view of the article from FIGS. 1 and 2, in the same position as in FIG. 1, the tool being located in the exposed or extracted working position,

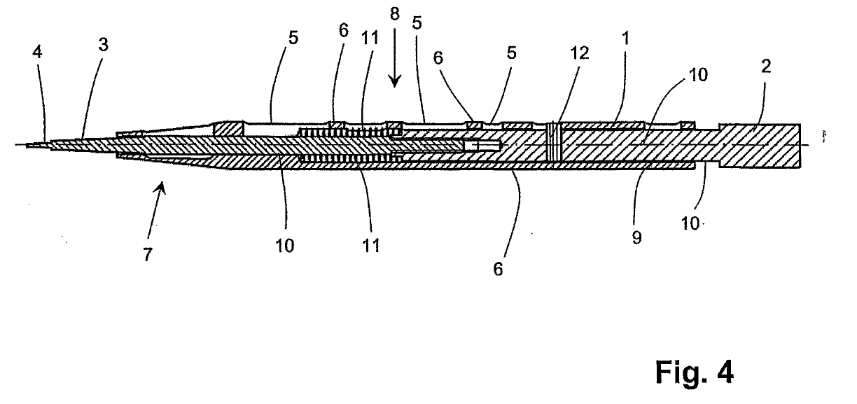

FIG. 4 shows a schematic view of the article from FIG. 3, in cross section and rotated through 90° about the longitudinal axis relative to FIG. 3, and

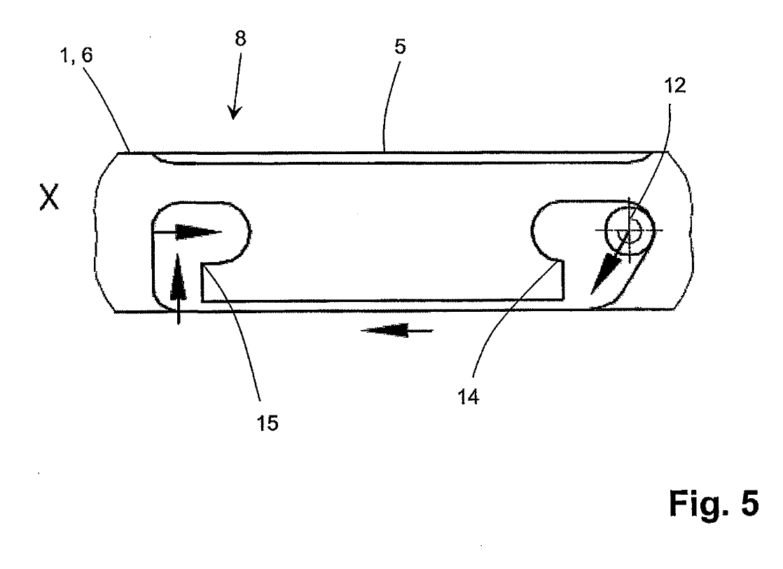

FIG. 5 shows an enlarged view of an area of the hand-held medical instrument from FIGS. 1 to 4, namely with a guide pin of the plunger located in the guide formed in the housing, together with a catch.

An illustrative embodiment of a hand-held medical instrument according to the invention is shown in a schematic view in FIG. 1, specifically a cutting instrument for ophthalmology. The cutting instrument comprises a housing 1 serving as a grip, and a tool holder 3 that can be displaced in the housing 1 via an actuating member 2. A diamond is used as the tool 4. The tool 4 can be displaced from a storage position in the housing 1, as shown in FIGS. 1 and 2, to a working position outside of the housing 1, as shown in FIGS. 3 and 4, by actuation of the actuating member 2.

According to the invention, the housing 1 has openings 5 in the housing wall 6, at least in the area of the tool 4 located in the storage position.

FIGS. 1 to 4 together show that the openings 5 are formed along the entire length of the housing 1. The figures also show that the housing 1 has a housing tip 7 that tapers continuously toward the free end, and a cylindrical housing shaft 8. The openings 5 are formed both in the housing tip 7 and also in the housing shaft 8.

The openings 5 are formed about the entire circumference of the housing 1, specifically aligned one behind another in the axial direction. In addition, the openings 5 have an oval shape with rounded edge areas. The openings 5 are formed close to one another such that the housing wall 6 is presented as a housing cage.

FIGS. 2 and 4 show particularly clearly that the housing 1 has a cylindrical passage 9 extending as far as the housing tip 7. A plunger 10 carrying the tool holder 3 is arranged displaceably inside the passage 9. It is also important that the plunger 10 tapers toward the tool holder 3. In the area of the taper, i.e. in the front area, namely between the plunger 10 and the housing 1, a compression spring 11 is arranged against whose force the tool 4 can be brought into the working position.

The plunger 10 is guided in the cylindrical area of the housing 1, i.e. in the housing shaft 8, and the tapered area of the plunger 10 is guided in the transition between the cylindrical area of the housing 1 and the housing tip 7, such that the plunger 10 is provided overall with optimal guiding, i.e. secure against tilting.

FIGS. 1, 3 and 5 show clearly that the plunger 10 is assigned an orthogonally protruding guide pin 12, which runs in a guide 13 formed in the housing 1. The guide pin 12 is screwed into the plunger 10.

The guide 13 is designed as a slit in the housing wall 6.

FIGS. 1, 3 and 5 also clearly show that the guide 13 or slit has a rear catch 14 against the tool 4 inadvertently being pushed out into the working position. Moreover, a front catch 15 is provided against the tool 4 inadvertently being pushed into the storage position. Both catches 14, 15 are designed in such a way that, by turning the plunger 10, namely via the actuating member 2, the guide pin 12 can be turned, in a suitable axial position, out of the locked position, namely out of the rear catch 14, and, after the tool has been pushed out by axial actuation of the actuating member 2, into a locked position, namely into the front catch 15, and can be locked or anchored there. The respective release from the catches 14, 15 accordingly takes place in reverse.

As regards other features that cannot be discerned from the figures, reference is made to the general part of the description, in order to avoid repetition.

Finally, it will be noted that the illustrative embodiment discussed above serves simply as an example to explain the hand-held instrument according to the invention, and said instrument is not limited to the illustrative embodiment.

1-23. (canceled)

24. A hand-held medical instrument, namely a surgical cutting instrument for use in ophthalmology, the instrument comprising:
a housing structured to serve as a grip;
a knife;
a tool holder, said tool holder being structured to receive said knife;
an actuating member in operable communication with said tool holder and said housing, wherein said tool holder is received within said housing so as to be displaceable from said housing via said actuating member, said knife being able to be displaced from a storage position in said housing to a working position outside of said housing by actuation of said actuating member, and wherein said housing defines openings therein at least in the area of said knife located in the storage position.

25. The hand-held instrument as claimed in claim 24, further comprising said housing defining openings therein along the entire length of said housing.

26. The hand-held instrument as claimed in claim 24, wherein said housing defines a free end and a housing tip that tapers continuously toward said free end, wherein said housing further defines a cylindrical housing shaft, and wherein said housing tip and said housing shaft define openings.

27. The hand-held instrument as claimed in claim 24, wherein said housing defines the openings around the circumference of said housing.

28. The hand-held instrument as claimed in claim 24, wherein said openings are offset relative to one another in the axial direction of said housing.

29. The hand-held instrument as claimed in claim 24, wherein said openings are in alignment one behind another in the axial direction of said housing.

30. The hand-held instrument as claimed in claim 24, wherein said openings comprise a substantially round configuration.

31. The hand-held instrument as claimed in claim 24, wherein said openings comprise an oval shape.

32. The hand-held instrument as claimed in claim 24, wherein said openings are designed in such a way that said housing forms a housing cage.

33. The hand-held instrument as claimed in claim 24, wherein said housing defines a cylindrical passage, and wherein the instrument further defines a plunger, said plunger being structured to carry said tool holder within said cylindrical passage as said tool holder is displaced within said housing.

34. The hand-held instrument as claimed in claim 33, wherein said plunger tapers toward said tool holder via a step formation, and wherein the instrument further comprises a compression spring positioned in the area of the taper between said plunger and said housing, said compression spring being biased against said tool holder to thereby urge said knife into the working position.

35. The hand-held instrument as claimed in claim 34, wherein said plunger is guided within said cylindrical passage of said housing, and said tapered area of said plunger is guided within the transition between said cylindrical passage of said housing and said housing tip or in said housing tip.

36. The hand-held instrument as claimed in claim 33, wherein said plunger defines an orthogonally protruding guide pin, said housing defines a guide, and wherein said guide pin runs in said guide formed in said housing.

37. The hand-held instrument as claimed in claim 36, wherein said guide pin is screwed into said plunger.

38. The hand-held instrument as claimed in claim 36, wherein said guide is configured as a slit in said housing.

39. The hand-held instrument as claimed in claim 38, wherein said housing defines a rear catch in operable communication with said slit to thereby prevent said knife inadvertently being pushed out into the working position, and wherein said housing defines a front catch structured to prevent said knife being inadvertently pushed into the storage position.

40. The hand-held instrument as claimed in claim 39, wherein said actuator member is structured to turn said plunger to thereby turn said guide pin from a locked position to an unlocked position and from an unlocked position to a locked position.

41. The hand-held instrument as claimed in one of claims 33, wherein said actuating member is made thicker or widened relative to said plunger, and wherein said actuating member defines opposite flattened surfaces.

42. The hand-held instrument as claimed in claim 33, wherein the tapered area of said plunger, said actuating member and said tool holder are made in one piece.

43. The hand-held instrument as claimed in claim 24, wherein said tool holder comprises a seat for said knife, into which seat said knife is adhesively bonded.

44. The hand-held instrument as claimed in claim 24, wherein said knife comprises a diamond blade.

45. The hand-held instrument as claimed in claim 33, wherein said housing, said plunger, said actuating member and said tool holder are made of stainless steel.

46. The hand-held instrument as claimed in claim 33, wherein said housing, said plunger, said actuating member and said tool holder are made of titanium or of a titanium alloy.
